(19) United States
(12) Patent Application Publication
(10) Pub. No.: US 2018/0103992 A1
(43) Pub. Date: Apr. 19, 2018

(54) METHOD OF TREATING MIGRAINE HEADACHE USING ULTRASOUND AND ELECTROCAUTERY OR RADIOFREQUENCY ABLATION

(71) Applicant: Bahman Guyuron, Lyndhurst, OH (US)

(72) Inventor: Bahman Guyuron, Lyndhurst, OH (US)

(21) Appl. No.: 15/784,329

(22) Filed: Oct. 16, 2017

Related U.S. Application Data
(60) Provisional application No. 62/408,980, filed on Oct. 17, 2016.
(60) Provisional application No. 62/408,184, filed on Oct. 14, 2016.

(51) Int. Cl. A61B 18/14; A61N 1/04; A61B 8/08

(52) U.S. Cl. CPC A61B 18/14; A61N 7/02; A61B 8/488; A61N 1/0456

(57) ABSTRACT

A method for treating headaches caused by a blood vessel irritating an associated nerve. The method is performed using a doppler ultrasound device and a treatment device (e.g., electrocautery or radiofrequency ablation). The method includes receiving an estimated location of an origin of the headaches. The estimated location is investigated for a blood vessel using the doppler ultrasound device. When the investigation of the estimated location identifies a blood vessel, the estimated location is cauterized or radiofrequency ablated using the treatment device, such that pressure is reduced on a nerve associated with the identified blood vessel and such that the treatment device does not directly stimulate the nerve.

### RELATED APPLICATIONS

This application claims the benefit of Application No. 62/408,980 filed on Oct. 17, 2016 and Application No. 62/408,184 filed on Oct. 14, 2016. Which are both herein incorporated by reference in their entirety.

### TECHNICAL FIELD

The present disclosure relates generally to treatment of migraine headaches and more particularly to treatment of migraine headache trigger sites using ultrasound and electrocautery or radiofrequency ablation.

### BACKGROUND

Migraine headache is a common and often debilitating neurological condition. A number of surgical techniques have been developed targeting sensory nerves in the head and neck that are thought to play a role in the initiation of migraine headaches. Several clinical studies have verified the efficacy and safety of these surgical techniques, which can result in significant improvement or elimination of migraine headaches in nearly 90 percent of patients, with minimal operative complications. Those surgical interventions target four major and several minor trigger sites that are shown in FIG. 1. The frontal trigger site (site I) involves the supraorbital and supratrochlear nerves, which are irritated by the glabellar muscles, the surrounding vessels, foramina, and fascial bands. In the temporal trigger site (site II), the zygomaticotemporal branch of the trigeminal nerve is compressed by the temporalis muscle and the tight deep temporal fascia, or irritated by the accompanying vessels. In the rhinogenic trigger site (site III), contact points between the septum, turbinates and concha bullosa, or sinus inflammation can irritate the terminal branches of the trigeminal nerve, triggering migraine headaches. In the fourth major site (site IV), the greater and/or the third occipital nerves are irritated by the semispinalis capitis muscle, fascial bands, and/or the occipital artery. The minor trigger sites consist of the auriculotemporal nerve (site V), which can be irritated by branches of the superficial temporal artery and fascial bands, the lesser occipital nerve (site VI), which can similarly be compressed by fascial bands and the occipital artery branches, and terminal branches of each of these main nerves.

Despite its high efficacy, a small number of patients who undergo migraine surgery do not have improvement in their migraine headaches. This unfavorable response in some patients has been ascribed to incomplete detection of all of the trigger sites and inadequate decompression of the primary trigger point. When a nerve has multiple compression or irritation points along its course, incomplete relief can be avoided by precise knowledge of the anatomy of the sensory nerves of the head and neck. The compression or irritation points of the frontal, temporal, occipital, and intranasal trigger points have been well described in several detailed anatomical studies.

### SUMMARY

The most common reason for failure of migraine surgery is the presence of multiple trigger sites, which may be masked by a more dominant pain site, thus resulting in failure of detection of all of the trigger sites. With the discovery that not all compression points are muscular, and with the development of new diagnostic modalities, a broader algorithm is needed.

Identification of trigger sites is essential to treatment of migraine headaches. Each patient's constellation of symptoms can point toward one or multiple trigger points. In the described treatment method, the patient is asked to point to the most frequent site from which migraine headaches originate (e.g., with one fingertip), and then the site is explored with a Doppler ultrasound. If an arterial Doppler signal is identified at the site, it is considered an active blood vessel trigger site. Once a blood vessel trigger site has been identified through this reliable and objective tool, electrocautery and/or radiofrequency ablation is performed on the blood vessel. Ablation/cauterization of the blood vessel reduces pressure on an associated nerve that is responsible for triggering the migraine headache, which results in reduction or elimination of migraine headache symptoms without invasive surgery.

According to one aspect, there is provided a method for treating headaches caused by a blood vessel irritating an associated nerve. The method is performed using a doppler ultrasound device and an treatment device. The method includes investigating an estimated location of an origin of the headaches for a blood vessel using the doppler ultrasound device. When the investigation of the estimated location identifies a blood vessel, the treatment device is used to cauterize and/or radiofrequency ablate the identified blood vessel, such that pressure is reduced on a nerve associated with the identified blood vessel and such that the treatment device does not directly stimulate the nerve.

Alternatively or additionally, the estimated location of the origin of the headaches comprises a location on a surface of a head of a patient.

Alternatively or additionally, a blood vessel is identified when an arterial doppler signal is detected by the doppler ultrasound device.

Alternatively or additionally, cauterizing and/or radiofrequency ablating the estimated location comprises performing radiofrequency ablation of the estimated location.

Alternatively or additionally, the estimated location of the origin of the headaches comprises a plurality of estimated locations.

Alternatively or additionally, each of the plurality of estimated locations are investigated using the doppler ultrasound device and, for each of the plurality of estimated locations at which a blood vessel is identified by the investigation, cauterization and/or radiofrequency ablation is performed using the treatment device.

Alternatively or additionally, a new estimated location of the origin of the headaches is received following either investigation of the estimated location when a blood vessel is not identified or alternatively after cauterization and/or radiofrequency ablation when a blood vessel is identified. The new estimated location is investigated for a blood vessel using the doppler ultrasound device. When the investigation of the estimated location identifies a blood vessel, the new estimated location is cauterized and/or radiofrequency ablated using the treatment device.

Alternatively or additionally, the method also includes, prior to cauterizing and/or radiofrequency ablating the estimated location, investigating the estimated location for an associated nerve. When the investigation of the estimated location identifies a blood vessel and an associated nerve, the estimated location is only cauterized and/or radiofrequency ablated using the treatment device when the identified blood vessel is sufficiently close to the associated nerve to be the cause of the headaches.

Alternatively or additionally, the identified blood vessel is sufficiently close to the associated nerve to be the cause of the headaches if the blood vessel is within 5 millimeters or 10 millimeters of the associated nerve.

Alternatively or additionally, the investigating of the estimated location for an associated nerve using ultrasound is performed using either the doppler ultrasound device or an ultrasound device that is separate from the doppler ultrasound device.

According to another aspect, there is provided a system for treating headaches caused by a blood vessel irritating an associated nerve. The system includes a doppler ultrasound device and an treatment device. The doppler ultrasound device is configured to investigate for a presence of a blood vessel using ultrasound at an estimated location of an origin of the headaches. The treatment device is physically separate from the doppler ultrasound device and is configured to cauterize and/or radiofrequency ablate the identified blood vessel when the investigation of the estimated location identifies a blood vessel, such that pressure is reduced on a nerve associated with the identified blood vessel.

Alternatively or additionally, the doppler ultrasound device is further configured to investigate using ultrasound the estimated location for a presence of a nerve causing the headaches.

According to a further aspect, there is provided a device for treating headaches caused by a blood vessel irritating an associated nerve. The device includes a doppler ultrasound transducer configured to investigate for a presence of a blood vessel using ultrasound at an estimated location of an origin of the headaches. The electrocautery source and/or radiofrequency source is physically connected to the doppler ultrasound transducer such that a focal point of the electrocautery source and/or radiofrequency source is focused on the estimated location when the doppler ultrasound transducer is positioned to investigate the estimated location. The electrocautery source and/or radiofrequency source is configured to cauterize and/or radiofrequency ablate the estimated location when the investigation of the estimated location identifies a blood vessel, such that pressure is reduced on a nerve associated with the identified blood vessel.

Alternatively or additionally, the device also includes a controller configured to cause the electrocautery source and/or radiofrequency source to perform cauterization and/or radiofrequency ablation of the estimated location simultaneously with the doppler ultrasound transducer imaging the estimated location.

Alternatively or additionally, the device further includes a controller configured to cause the electrocautery source and/or radiofrequency source to perform cauterization and/or radiofrequency ablation of the estimated location sequentially after investigation of the estimated location using the doppler ultrasound transducer.

Alternatively or additionally, the doppler ultrasound transducer is further configured to investigate using ultrasound the estimated location for a presence of a nerve causing the headaches.

A number of features are described herein with respect to embodiments of the invention; it will be appreciated that features described with respect to a given embodiment also may be employed in connection with other embodiments.

The following description and the annexed drawings set forth certain illustrative embodiments of the invention. These embodiments are indicative, however, of but a few of the various ways in which the principles of the invention may be employed. Other objects, advantages and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention in which similar reference numerals are used to indicate the same or similar parts in the various views.

FIG. 1 is a schematic view of the four major and several minor trigger sites.

FIG. 2 is an image of Doppler ultrasound being used at the temporal triggering site.

FIGS. 3A and 3B are diagrams of a treatment device.

FIG. 4 is a flow diagram of a method for treating headaches using the treatment device.

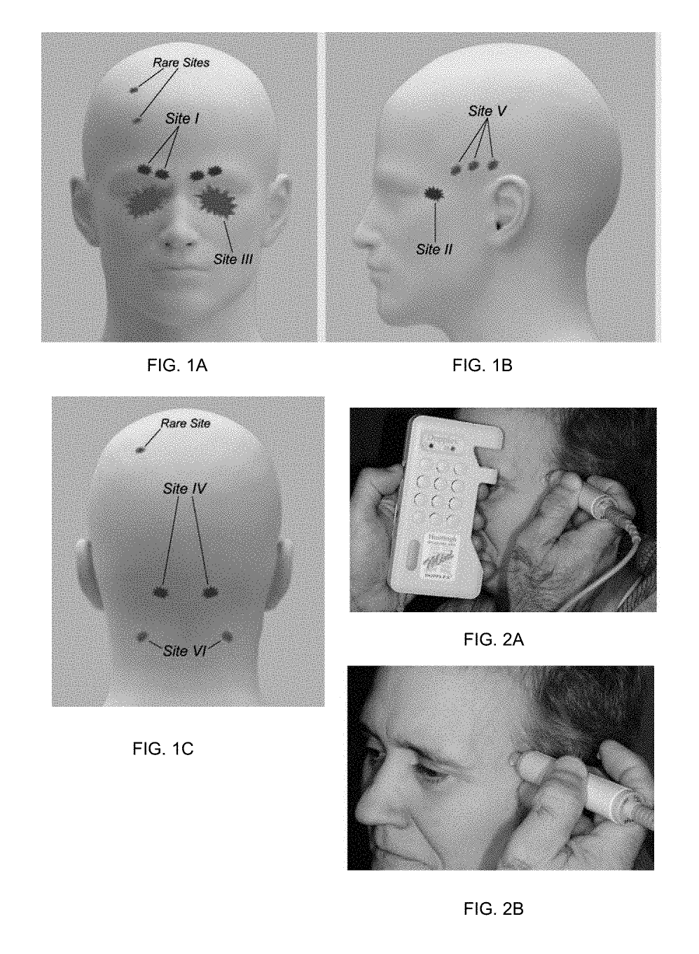

### DETAILED DESCRIPTION

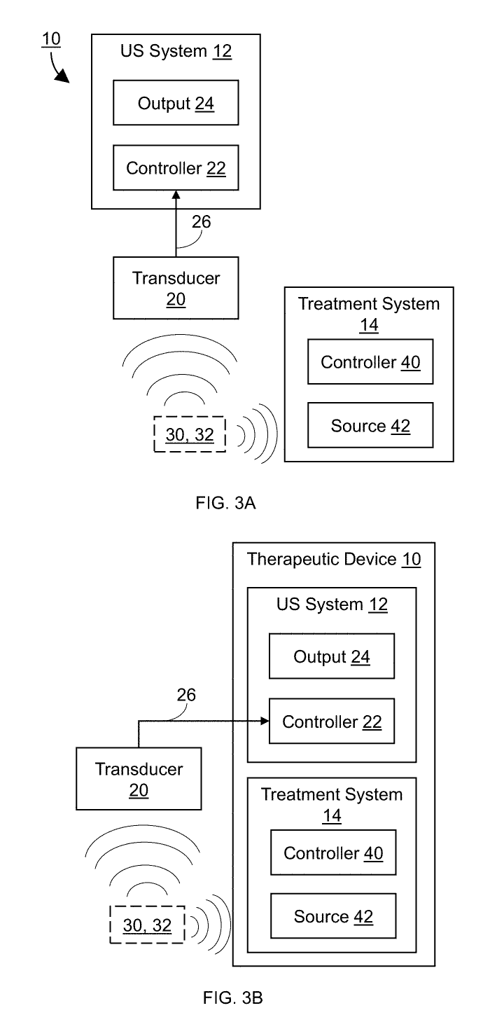

A treatment device 10 is shown in FIGS. 3A and 3B and a method of treating migraine headaches is described below. The method may be used to treat nummular headaches and any type of headache where a vessel is irritating an adjacent nerve. The treatment device 10 includes a Doppler ultrasound system 12 and a treatment system 14 (e.g., an electrocautery system and/or a radiofrequency ablation system). As is shown in FIG. 3A, the ultrasound system 12 and the treatment system 14 may be separate devices that are used sequentially or simultaneously, e.g., after injection of small amount of local anesthetic that causes vasoconstriction. Alternatively, as is shown in FIG. 3B, the ultrasound system 12 and the treatment system 14 may comprise a single integrated unit.

A user may use the Doppler ultrasound system 12 to identify trigger sites for migraine headaches. For example, a patient may be asked to point toward one or more trigger points from which migraine headaches have previously originated. The area identified by the patient may then be explored with Doppler ultrasound using the Doppler ultrasound system 12.

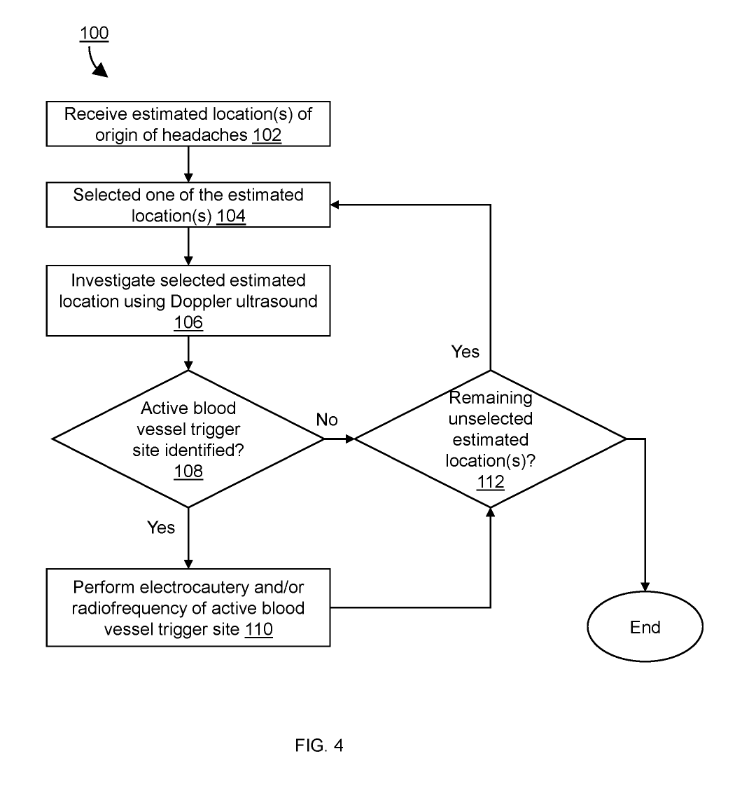

Several of the nerves implicated in migraine headache pathogenesis may intersect with an artery, including the supraorbital and supratrochlear nerves and their branches, the auriculotemporal nerve and its branches, the zygomaticotemporal nerve and its branches, the greater occipital nerve, the lesser occipital nerve, and even the zygomaticofacial nerve. Patients who have such an intersection may complain of pulsatile, throbbing headache. Doppler ultrasound is used to identify trigger points where an artery is irritating the nerve (see FIG. 4). Doppler ultrasound is able to pick up an arterial signal at the location of pain identified by the patient. Doppler ultrasound may also be used to identify the location of the nerve.

The Doppler ultrasound system 12 includes an ultrasound transducer 20, a controller 22, and an output 24. The ultrasound transducer 20 emits and detects ultrasound signals. In particular, the ultrasound transducer 20 emits ultrasound focused at a particular location 30 and detects ultrasound returning from this particular location 30. The ultrasound transducer 20 outputs a signal 26 representing the ultrasound waves detected by the transducer 20. The controller 22 controls emission of ultrasound signals by the ultrasound transducer 20 and receives from the ultrasound transducer 20 the output signal 26. The controller 22 receives the output signal 26 and outputs to the output device 24 an indication of blood flow in particular location 30. The output device 24 may comprise a speaker, displayer screen, or any suitable device for providing information to a user. For example, the returning Doppler ultrasound signal may be output as sound from the output device 24. For example, the sound output by the output device 24 may be used to differentiate between an arterial blood vessel and a venous blood vessel.

If an arterial Doppler signal is identified at the particular location 30 (e.g., the area identified by the patient), then the particular location 30 may be considered an active blood vessel trigger site. Upon detecting a blood vessel trigger site, the treatment system 14 is used to ablate the active blood vessel trigger site.

Ultrasound may also be used to detect the nerve and/or the blood vessel causing the migraine headache. That is, ultrasound may be used to detect the nerve being triggered by the blood vessel, as well as the triggering blood vessel. Detecting the nerve using ultrasound may be performed using an ultrasound device (e.g., an imaging ultrasound device) separate from the ultrasound device used to emit the doppler ultrasound signal (e.g., the doppler ultrasound device). Alternatively, detecting the nerve using ultrasound may be performed using the same ultrasound device used to emit the doppler ultrasound signal.

By imaging the nerve as well as the blood vessel, it can be assured that blood vessels unrelated to the nerve are not mistakenly ablated with radiofrequency energy. For example, if a blood vessel is located near a nerve (but the blood vessel is not located close enough to the nerve to cause a migraine headache), then this blood vessel may be left alone and the search for the blood vessel triggering the nerve may continue or for the nerve that is being stimulated or irritated by the muscle, fascia or any other element that is identified with the ultrasound and is treated with the radiofrequency ablation or electrocautery. For example, a blood vessel may be considered close enough to a nerve to cause a migraine headache if the blood vessel is less than 3 millimeters (mm), less than 5 mm, less than 10 mm, or less than 15 mm from the nerve.

Electrocautery and radiofrequency ablation are medical procedure in which a tissue is ablated using heat generated from alternating current. By applying (e.g., radio) energy to a blood vessel, the targeted blood vessel may be closed. Blood vessels identified as causing migraine headaches often interact with nerves, such that the blood vessel applies pressure on the nerve. This pressure may be the cause of migraine headaches. By using electrocautery and/or radiofrequency ablation to close a blood vessel, pressure is reduced on the nerve, mitigating or eliminating the symptoms of migraine headaches.

Electrocautery and radiofrequency ablation allows migraine headaches to be treated without directly stimulating the nerve that is causing the migraine headaches and often without the need for general anesthetic. Also, using electrocautery or radiofrequency ablation in this manner specifically treats the desired blood vessel without significant collateral damage (e.g., to adjacent tissues).

The treatment system 14 includes a controller 40 and a source 42. The controller 40 directs the source 42 to emit radio energy at a particular location 32. The particular location 32 of the treatment system 14 and the particular location 30 of the Doppler ultrasound system 12 may be made to overlap, such that when an active blood vessel trigger site is identified at the particular location 30 of the Doppler ultrasound system 12, the treatment system 14 may be used to cauterize and/or radiofrequency ablate the active blood vessel trigger site. For example, the Doppler ultrasound system 12 and the treatment system 14 may be physically joined (as shown in FIG. 3B) such that the particular location 30 of the Doppler ultrasound system 12 and the particular location 32 of the treatment system 14 overlap. Alternatively, a user may first use the Doppler ultrasound system 12 to identify an active blood vessel trigger site based on the known relationship between the ultrasound transducer 20 and the particular location 30 of the Doppler ultrasound system 12. Next, a user may position the treatment system 14 such that the particular location 32 of the treatment system 14 overlaps with the active blood vessel trigger site. The user may position the treatment system 14 based on a known location of the particular location 32 relative to the treatment system 14.

A method 100 for treating headaches is shown in FIG. 4. In process block 102 an estimated location(s) of headache origination is received. In process block 104, one of the estimated location(s) is selected. In process block 106, the selected estimated location is investigated using Doppler ultrasound as described above. If the selected estimated location is identified as an active blood vessel trigger site in decision block 108, electrocautery and/or radiofrequency ablation is performed on the active blood vessel trigger site in process block 110. If the selected estimated location is not an active blood vessel trigger site or after performing electrocautery/radiofrequency ablation, in decision block 112 a check is made to determine if there are any remaining received estimated location(s) that have not yet been selected and investigated using ultrasound. If there are remaining unselected estimated location(s) then processing returns to process block 104. As will be understood by one of ordinary skill in the art, as opposed to receiving a number of estimated locations, the method may also operate by receiving a single estimated location for the origin of headaches, investigating this location, and then receiving another estimated location.

Although the invention has been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A method for treating headaches caused by a blood vessel irritating an associated nerve, the method performed using a doppler ultrasound device and an treatment device, the method comprising:
using the doppler ultrasound device, investigating an estimated location of an origin of the headaches for a blood vessel;
when the investigation of the estimated location identifies a blood vessel, cauterizing or radiofrequency ablating the identified blood vessel using the treatment device, such that pressure is reduced on a nerve associated with the identified blood vessel and such that the treatment device does not directly stimulate the nerve.

2. The method of claim 1, wherein the estimated location of the origin of the headaches comprises a location on a surface of a head of a patient.

3. The method of claim 1, wherein a blood vessel is identified when an arterial doppler signal is detected by the doppler ultrasound device.

4. The method of claim 1, wherein cauterizing or radiofrequency ablating the estimated location comprises performing radiofrequency ablation of the estimated location.

5. The method of claim 1, wherein the estimated location of the origin of the headaches comprises a plurality of estimated locations.

6. The method of claim 5, wherein:
each of the plurality of estimated locations are investigated using the doppler ultrasound device; and
for each of the plurality of estimated locations at which a blood vessel is identified by the investigation, cauterization or radiofrequency ablation is performed using the treatment device.

7. The method of claim 1, wherein:
a new estimated location of the origin of the headaches is received following either investigation of the estimated location when a blood vessel is not identified or alternatively after cauterization or radiofrequency ablation when a blood vessel is identified;
the new estimated location is investigated for a blood vessel using the doppler ultrasound device; and
when the investigation of the estimated location identifies a blood vessel, cauterizing or radiofrequency ablating the new estimated location using the treatment device.

8. The method of claim 1, further comprising:
prior to cauterizing or radiofrequency ablating the estimated location, investigating the estimated location for an associated nerve using ultrasound; and
when the investigation of the estimated location identifies a blood vessel and an associated nerve, only cauterizing or radiofrequency ablating the estimated location using the treatment device when the identified blood vessel is sufficiently close to the associated nerve to be the cause of the headaches.

9. The method of claim 8, wherein the identified blood vessel is sufficiently close to the associated nerve to be the cause of the headaches if the blood vessel is within 5 millimeters or 10 millimeters of the associated nerve.

10. The method of claim 8, wherein the investigating of the estimated location for an associated nerve using ultrasound is performed using either the doppler ultrasound device or an ultrasound device that is separate from the doppler ultrasound device.

11. A system for treating headaches caused by a blood vessel irritating an associated nerve, the system comprising:
a doppler ultrasound device configured to investigate for a presence of a blood vessel using ultrasound at an estimated location of an origin of the headaches; and
a treatment device that is physically separate from the doppler ultrasound device and is configured to cauterize or radiofrequency ablate the identified blood vessel when the investigation of the estimated location identifies a blood vessel, such that pressure is reduced on a nerve associated with the identified blood vessel.

12. The system of claim 11, wherein the doppler ultrasound device is further configured to investigate using ultrasound the estimated location for a presence of a nerve causing the headaches.

13. A device for treating headaches caused by a blood vessel irritating an associated nerve, the device comprising:
a doppler ultrasound transducer configured to investigate for a presence of a blood vessel using ultrasound at an estimated location of an origin of the headaches; and
an electrocautery source and/or a radiofrequency source that is physically connected to the doppler ultrasound transducer such that a focal point of the electrocautery source and/or radiofrequency source is focused on the estimated location when the doppler ultrasound transducer is positioned to investigate the estimated location;
wherein the electrocautery source and/or radiofrequency source is configured to cauterize and/or radiofrequency ablate the estimated location when the investigation of the estimated location identifies a blood vessel, such that pressure is reduced on a nerve associated with the identified blood vessel.

14. The device of claim 13, further comprising a controller configured to cause the electrocautery source and/or radiofrequency source to perform cauterization and/or radiofrequency ablation of the estimated location simultaneously with the doppler ultrasound transducer imaging the estimated location.

15. The device of claim 13, further comprising a controller configured to cause the electrocautery source and/or radiofrequency source to perform cauterization or radiofrequency ablation of the estimated location sequentially after investigation of the estimated location using the doppler ultrasound transducer.

16. The device of claim 13, wherein the doppler ultrasound transducer is further configured to investigate using ultrasound the estimated location for a presence of a nerve causing the headaches.
